(19) United States
(12) Patent Application Publication
(10) Pub. No.: US 2012/0219598 A1
(43) Pub. Date: Aug. 30, 2012

(54) POLYMERIC MICELLES FOR REDUCING LDL IN VIVO

(76) Inventors: Kathryn E. Uhrich, New Brunswick, NJ (US); Prabhas Moghe, New Brunswick, NJ (US); Sarah Sparks, New Brunswick, NJ (US); Sarah Hehir, Durham (GB); Li Gu, New Brunswick, NJ (US); Nicole Iverson, New Brunswick, NJ (US); Nicole Plourde, New Brunswick, NJ (US)

(21) Appl. No.: 13/402,694

(22) Filed: Feb. 22, 2012

Related U.S. Application Data
(60) Provisional application No. 61/445,382, filed on Feb. 22, 2011.

(51) Int. Cl. A61K 31/25; C12N 5/071; A61P 9/10; C07C 69/76; A61K 9/00

(52) U.S. Cl. 424/400; 560/81; 514/533; 435/375

(57) ABSTRACT

The invention encompasses micelle assemblies, compositions having micelle assemblies, and methods for preparing micelle assemblies and compositions thereof. The invention also includes compounds of the formula I:

A-X—Y—Z—R₁ (I)

The invention includes methods of encapsulating molecules using the compounds of the invention.

### RELATED APPLICATION

This application claims benefit of U.S. Provisional Application Ser. No. 61/445,382 filed on Feb. 22, 2011, which application is herein incorporated by reference.

### GOVERNMENT FUNDING

The invention described herein was made with government support under Grant Number R21 HL093753 awarded by the National Institutes of Health. The United States Government has certain rights in the invention.

### BACKGROUND OF THE INVENTION

Polymeric micelles are self-assembled amphiphilic block copolymers. These micelles have attracted attention as promising colloidal drug delivery systems (V. P. Torchilin J. Controlled. Release. 2001, 73, 137; C. Allen, D. et al., Colloids and Surfaces B: Biointerfaces 1999, 16, 3; and H. Otsuka, et al., Current Opinion in Colloid & Interface Science 2001, 6, 3). In these colloidal systems, the hydrophobic block typically forms the core, essentially a “microcontainer” for a lipophilic pharmaceutical (K. Kataoka, et al., Adv. Drug Delivery Rev. 2001, 47, 113). The hydrophilic part forms the outer shell, stabilizing the interface between the core and the external aqueous environment. Compared to traditional micellar systems, these polymeric surfactant-based drug carriers display apparent advantages such as lower critical micelle concentration (CMC), improved bioavailability, reduction of toxicity, enhanced permeability across the physiological barriers, and substantial changes in drug biodistribution.

Amphiphilic star-like macromolecules (ASMs) have also been studied for drug delivery applications. (See, e.g., U.S. patent application Ser. No. 09/298729 filed Apr. 23, 1999; U.S. patent application Ser. No. 09/422,295, filed Oct. 21, 1999, and International Patent Application US00/10050 filed Apr. 18, 2000). The core-shell, amphiphilic structure of ASMs is covalently linked, which makes it thermodynamically stable compared to conventional micellar systems. Thus, ASM's offer numerous advantages over conventional micellar systems. Despite these advantages, the use of ASM's is somewhat limited due to the difficulty and cost associated with their preparation. Accordingly, there is a need for additional micellar systems that possess some of the advantages associated with the thermodynamic stability of ASM's, but which are easier and less expensive to prepare.

### SUMMARY OF THE INVENTION

Applicant has discovered that compounds of formula (I):

A-X—Y—Z—R₁ (I)

wherein

A is an anionic group or is absent;

X is aryl or heteroaryl, wherein any aryl or hetereoaryl of X is substituted with one or more (e.g., 1, 2, 3, 4, 5 or 6) groups selected from hydroxy and amine, wherein one or more (e.g., 1, 2, 3, 4, 5 or 6) of the hydroxy or amine groups is acylated with a hydrophobic chain, and wherein any aryl or hetereoaryl of X is optionally substituted with one or more (e.g. 1, 2, 3, 4, 5 or 6) groups selected from halo, carboxy, CN, NO₂, N((C₁-C₆)alkyl)₂, (C₁-C₆)alkyl, (C₁-C₆)alkoxy, CF₃ and OCF₃;

Y is —C(═O)—, —C(═S)—, or is absent;

Z is O, S or NH; and

R₁ is a polyether.

or a salt thereof; will aggregate in a solvent to form micellar structures.

These aggregates are useful in drug delivery applications, as well as in many other applications where traditional micelles and ASM's can be applied. The aggregates formed from compounds of formula (I), can be prepared without much of the difficulty and cost associated with the preparation of ASM's. Accordingly, the invention provides a compound of formula (I) as described above. Such compounds of formula (I) are useful intermediates for preparing aggregates that can be used in drug delivery applications.

The invention also provides a composition comprising a plurality of compounds of formula (I) in a solvent. Such a composition is useful for preparing aggregates comprising compounds of formula (I). The invention also provides a composition comprising a plurality of compounds of formula (I) in a solvent, wherein the compounds of formula (I) are associated into one or more aggregates.

The invention also provides an aggregate structure formed by combining a plurality of compounds of formula (I) as described herein in a solvent; and allowing the compounds to form the aggregate.

The invention also provides a method for preparing an aggregate structure of formula (I), as described herein, comprising combining a plurality of compounds of formula (I) in a solvent; and allowing them to form the aggregate structure.

The invention also provides an encapsulate comprising a molecule (e.g. a therapeutic agent) surrounded or partially surrounded by an aggregate of the invention.

The invention further provides a method where the aggregates are formed by combining a plurality of compounds of formula (I) in a solvent.

The invention also provides a method for preparing an encapsulate of the invention comprising combining a plurality of compounds of formula (I) and a molecule (e.g. a therapeutic agent) in a solvent, and allowing the compounds of formula (I) to aggregate around the molecule, to provide the encapsulate (i.e. the molecule surrounded or partially surrounded by a plurality of compounds of formula (I)).

The invention also provides a composition comprising a solvent, and an aggregate of a plurality of compounds of formula (I) surrounding a molecule (e.g., a therapeutic agent).

The invention also provides a pharmaceutical composition comprising an encapsulate of the invention (i.e. a therapeutic agent surrounded or partially surrounded by a plurality of compounds of formula (I)), or a pharmaceutically acceptable salt thereof, and a pharmaceutically acceptable carrier.

The invention also provides a method for delivering a therapeutic agent to an animal (e.g., a human) in need of treatment with the agent comprising administering an encapsulate of the invention, or a pharmaceutically acceptable salt thereof, comprising the agent to the animal.

The invention also provides a method for reducing LDL in a mammal (e.g., a human) comprising administering an effective amount of a compound of formula I as described herein, or a pharmaceutically acceptable salt thereof, to the mammal.

The invention also provides a method for preventing the uptake of LDL by a cell comprising contacting the cell with a compound of formula I as described in herein.

The invention also provides a method for inhibiting atherosclerosis or atherosclerotic development in a mammal (e.g., a human), comprising administering an anti-atherosclerosis or anti-atherosclerotic development amount of a compound of formula I as described herein, or a pharmaceutically acceptable salt thereof, to the mammal.

The invention also provides intermediates and processes useful for preparing compounds of formula (I) as described herein.

### BRIEF DESCRIPTION OF THE FIGURE

FIG. 1. Evaluation of AM through a) in vitro inhibition of oxLDL uptake. over 24 hr in HEK cells induced to express SR-A.

### DETAILED DESCRIPTION

A is an anionic group or is absent. When present, A may optionally be substituted with or attached to a bioactive or therapeutically active molecule. The bioactive or therapeutically active molecule can be any known to one of ordinary skill in the art such as those described below. In one embodiment, the bioactive or therapeutically active molecule includes, but is not limited to, vitamin E, sulfonic acids, sulfonates, or salicylic acid.

In another embodiment of the invention the bioactive or therapeutically active molecule can be attached to the R¹ group of a compound of formula I. The bioactive or therapeutically active molecule can be attached to the R¹ group through any suitable functional group. Suitable functional groups include but not limited to amides, esters, sulfonamides, sulfonates, anhydrides.

As used herein the term “aryl” refers to a ring structure of from about 6 to 14 carbon atoms in the ring. Aryl includes a single aromatic ring (e.g. phenyl). Aryl also includes a multiple condensed ring (e.g. a bicyclic or multicyclic ring) wherein all of the condensed rings may or may not be aromatic provided that at least one of the condensed rings is aromatic. Such bicyclic or multicyclic aryls may be optionally substituted with one or more (e.g. 1, 2 or 3) oxo groups on any non-aromatic ring of the condensed ring. It is to be understood that the point(s) of attachment of a bicyclic or multicyclic aryl can be at any position of the ring system including an aromatic or non-aromatic portion of the ring. Typical aryl groups include, but are not limited to phenyl, indenyl, naphthyl, 1, 2, 3, 4-tetrahydronaphthyl, anthracenyl, and the like.

The term “heteroaryl” as used herein refers to a ring structure from about 1 to 10 carbon atoms and 1 to 5 heteroatoms selected from the group consisting of oxygen, nitrogen and sulfur in the ring. The sulfur and nitrogen heteroatoms atoms may also be present in their oxidized forms. Heteroaryl includes a single aromatic ring with at least one heteroatom (e.g. pyridyl, pyrimidinyl or furyl). Heteroaryl also includes a multiple condensed ring (e.g. a bicyclic or multicyclic ring) wherein all of the condensed rings may or may not be aromatic or contain a heteroatom provided that at least one of the condensed rings is aromatic with at least one heteroatom (e.g. indolizinyl or benzothienyl). Such bicyclic or multicyclic heteroaryls may be optionally substituted with one or more (e.g. 1, 2 or 3) oxo groups on any non-aromatic portion of the condensed ring. It is to be understood that the point(s) of attachment of a bicyclic or multicyclic heteroaryl can be at any position of the ring system including an aromatic or non-aromatic portion of the ring. Typical heteroaryl groups include, but are not limited to pyridyl, pyrrolyl, pyrazinyl, pyrimidinyl, pyridazinyl, pyrazolyl, thienyl, indolyl, thiophenyl, imidazolyl, oxazolyl, thiazolyl, furyl, oxadiazolyl, thiadiazolyl, quinolyl, isoquinolyl, benzothiazolyl, benzoxazolyl, indazolyl, indolyl, quinoxalyl, quinazolyl, 5,6,7,8-tetrahydroisoquinoline and the like.

The term “amine” as used herein refers to The term “amine” also refers to —NHR_{b} wherein each R_{b} is (C₁-C₆)alkyl, —(C₁-C₆)alkylaryl or aryl.

The term “anionic group” refers to groups that are negatively charged or groups that are capable of supporting a negative charge. Anionic groups include but are not limited to carboxy (—CO₂H), —SO₃H, —NHSO₂R_{c}, PO₃H or NO₂ or salts thereof, wherein R_{c} is CF₃, (C₁-C₆)alkyl, —(C₁-C₆)alkylaryl or aryl. The salts of the anionic groups, including inorganic and organic salts, are readily known by those skilled in the art.

Additionally, administration of a compound of formula I as a pharmaceutically acceptable acid or base salt may be appropriate. For example, alkali metal (e.g. sodium, potassium or lithium) or alkaline earth metal (e.g. calcium) salts of carboxylic acids, sulfonic acids, sulfonamides or other anionic groups can be made. Examples of pharmaceutically acceptable salts also include organic acid addition salts formed with acids which form a physiological acceptable anion, for example, tosylate, methanesulfonate, acetate, citrate, malonate, tartarate, succinate, benzoate, ascorbate, a-ketoglutarate, and a-glycerophosphate. Suitable inorganic salts may also be formed, including hydrochloride, sulfate, nitrate, bicarbonate, and carbonate salts.

Halo refers to fluoro, chloro, bromo, or iodo.

Alkyl, alkene, alkyne, alkoxy, etc. denote both straight and branched groups; but reference to an individual radical such as propyl embraces only the straight chain radical, a branched chain isomer such as isopropyl being specifically referred to.

Alkene refers to a group that has one or more carbon-carbon double bonds.

Alkyne refers to a group that has one or more carbon-carbon triple bonds.

Alkoxy refers to the group —O-alkyl (e.g. a group wherein a alkyl radical is connected to a molecule through an oxygen atom).

Carboxy refers to —CO₂H.

The term “hydrophobic chain” refers to a chain comprising one or more (C₁-C₂₄)allcyl, (C₂-C₂₄)alkene, (C₂-C₂₄)alkyne or aryl groups or combinations thereof. The hydrophobic chain is generally carbon in nature and lacks polarity. The “hydrophobic chain” is capable of acylating the hydroxy or amine groups on the aryl or heteroaryl of X through moieties such as a carboxylate, sulfonate, phosphate, phosphonate, and the like. The term hydrophobic chain also includes fatty acids or fatty acid residues.

As used herein, the term fatty acid or fatty acid residue includes fatty acids and fatty oils as conventionally defined, for example, long-chain aliphatic acids that are found in natural fats and oils. Fatty acids typically comprise from about 2 to about 24 carbon atoms. Preferably, fatty acids comprise from about 6 to about 18 carbon atoms. The term “fatty acid” encompasses compounds possessing a straight or branched aliphatic chain and an acid group, such as a carboxylate, sulfonate, phosphate, phosphonate, and the like. The “fatty acid” compounds are capable of “esterifying” or forming a similar chemical linkage with hydroxy groups or amine groups on the aryl or heteroaryl of X. Examples of suitable fatty acids include caprylic, capric, lauric, myristic, myristoleic, palmitic, palmitoleic, stearic, oleic, linoleic, eleostearic, arachidic, behenic, erucic, and like acids. Fatty acids can be derived from suitable naturally occurring or synthetic fatty acids or oils, can be saturated or unsaturated, and can optionally include positional or geometric isomers. Many fatty acids or oils are commercially available or can be readily prepared or isolated using procedures known to those skilled in the art.

As used herein, the term polyether includes poly(alkylene oxides) having between about 2 and about 150 repeating units. Typically, the poly(alkylene oxides) have between about 50 and about 115 repeating units. The alkylene oxide units contain from 2 to 10 carbon atoms and may be straight chained or branched. Preferably, the alkylene oxide units contain from 2 to 10 carbon atoms. Poly(ethylene glycol) (PEG) is preferred. Alkoxy-, amino-, carboxy-, and sulfo-terminated poly(alkylene oxides) are preferred, with methoxy-terminated poly(alkylene oxides) being more preferred.

In one embodiment the polyether has the following structure:

R₅—(R₆—O—)ₐ—R₆—

wherein R₅ is a 1 to 20 carbon straight-chain or branched alkyl group, —OH, —OR₇, —NH₂, —NHR₇, —NHR₇R₈, —CO₂H, —SO₃H (sulfo), —CH₂—OH, —CH₂—OR₇, —CH₂—O—CH₂—R₇, —CH₂—NH₂, —CH₂—NHR₇, —CH₂—NR₇R₈, —CH₂CO₂H, —CH₂ SO₃H, or —O—C(═O)—CH₂—CH₂—C(═O)—O—;

R₆ is a 1 to 10 carbon straight-chain or branched divalent alkylene group;

each R₇ and R₈ is independently a 1 to 6 carbon straight-chain or branched alkylene group; and

a is an integer from 2 to 150, inclusive.

In certain embodiments, a is an integer from 20 to 140, inclusive. In certain embodiments, a is an integer from 50 to 130, inclusive. In certain embodiments, a is an integer from 75 to 130, inclusive. In certain embodiments, a is an integer from 100 to 130, inclusive. In certain embodiments, a is 113.

In another embodiment the polyether is methoxy terminated poly(ethylene glycol).

In a compound of formula (I), a poly(alkylene oxide) can be linked to an aryl or heteroaryl, for example, through an ether, thioether, amine, ester, thioester, thioamide, or amide linkage. Preferably, a poly(alkylene oxide) is linked to an aryl or heteroaryl by an ester or amide linkage in a compound of formula (I).

A specific value for X is an aryl, wherein the aryl is substituted with two or more hydroxy groups, wherein one or more of the hydroxy groups is acylated with a hydrophobic chain, and wherein the aryl is optionally substituted with one or more groups selected from halogen, carboxy, CN, NO₂, N((C₁-C₆)alkyl)₂, (C₁-C₆)alkyl, (C₁-C₆)alkoxy, CF₃ and OCF₃.

Another specific value for X is phenyl substituted with two or more hydroxy groups, wherein two or more of the hydroxy groups are acylated with a hydrophobic chain.

Another specific value for X is an heteroaryl, wherein the heteroaryl is substituted with two or more hydroxy groups, wherein one or more of the hydroxy groups is acylated with a hydrophobic chain, and wherein the heteroaryl is optionally substituted with one or more groups selected from halogen, carboxy, CN, NO₂, N((C₁-C₆)alkyl)₂, (C₁-C₆)alkyl, (C₁-C₆)alkoxy, CF₃ and OCF₃.

A specific value for A is absent.

Another specific value for A is an anionic group.

Another specific value for A is carboxy, —SO₃H or —PO₃H.

Another specific value for A is carboxy.

A specific value for Y is —C(═O)—.

Another specific value for Y is —C(═S)—.

Another specific value for Y is absent.

A specific value for Z is O.

Another specific value for Z is S.

Another specific value for Z is NH.

A specific value for Y—Z is —C(═O)NH—, —C(═O)O— or —C(═S)O—.

Another specific value for Y—Z is —C(═O)NH— or —C(═O)O—.

Another specific value for Y—Z is —C(═O)O—.

A specific group of compounds of formula I are compounds wherein the polyether is a poly(alkylene oxide) having between about 2 and about 150 repeating units. In certain embodiments, the polyether is a poly(alkylene oxide) having between about 20 and about 140 repeating units. In certain embodiments, the polyether is a poly(alkylene oxide) having between about 50 and about 130 repeating units. In certain embodiments, the polyether is a poly(alkylene oxide) having between about 75 and about 130 repeating units. In certain embodiments, the polyether is a poly(alkylene oxide) having between about 100 and about 130 repeating units. In certain embodiments, the polyether is a poly(alkylene oxide) having about 113 repeating units.

A specific group of compounds of formula I are compounds wherein the alkylene oxide units contain from 2 to 4 carbon atoms and may be straight chained or branched.

A specific group of compounds of formula I are compounds wherein the polyether is alkoxy-terminated.

A specific group of compounds of formula I are compounds wherein the polyether has the following structure:

R₅—(R₆—O)ₐ—R₆—

wherein R₅ is a 1 to 20 carbon straight-chain or branched alkyl group, —OH, —OR₇, —NH₂, —NHR₇, —NHR₇R₈, —CO₂H, —SO₃H (sulfo), —CH₂—OH, —CH₂—OR₇, —CH₂—O—CH₂—R₇, —CH₂—NH₂, —CH₂—NHR₇, —CH₂—NR₇R₈, —CH₂CO₂H, —CH₂SO₃H, or —O—C(═O)—CH₂—CH₂—C(═O)—O—;

R₆ is a 1 to 10 carbon straight-chain or branched divalent alkylene group;

each R₇ and R₈ is independently a 1 to 6 carbon straight-chain or branched alkylene group; and

a is an integer from 2 to 150, inclusive.

In certain embodiments, a is an integer from 20 to 140, inclusive. In certain embodiments, a is an integer from 50 to 130, inclusive. In certain embodiments, a is an integer from 75 to 130, inclusive. In certain embodiments, a is an integer from 100 to 130, inclusive. In certain embodiments, a is 113.

A specific group of compounds of formula I are compounds wherein the polyether is a methoxy terminated poly(ethylene glycol).

In one embodiment, the hydrophobic chain comprises (C₁-C₂₄)alkyl, (C₂-C₂₄)alkene or (C₂-C₂₄)alkyne.

In another embodiment, the hydrophobic chain comprises (C₆-C₁₈)alkyl, (C6-C18)alkene or (C₆-C₁₈)alkyne.

Another specific value for hydrophobic chain is a fatty acid, wherein the fatty acid is caprylic, capric, lauric, myristic, myristoleic, palmitic, palmitoleic, stearic, oleic, linoleic, arachidic, behenic, or erucic acid, or a mixture thereof.

In certain embodiments, the compound of formula (I) is:

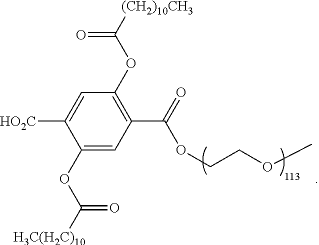

Certain embodiments of the invention provide a composition comprising a plurality of compounds of formula (I), as described herein, in a solvent, wherein the compounds of formula (I) form one or more aggregate structures.

In certain embodiments, the solvent comprises water.

In certain embodiments, the solvent is water.

As used herein, the term “aggregate” means a plurality of compounds of formula (I) in a solvent that have organized into an ordered structure, for example, a structure having a hydrophobic core and a surrounding hydrophilic layer, or a structure having a hydrophilic core and a surrounding hydrophobic layer.

As used herein, the term “a plurality of compounds of formula (I)” means more than one compound of formula (I). In such a plurality, each compound of formula (I) can have the same structure, or the plurality can include compounds of formula (I) that have differing structures. In a preferred embodiment, the term “a plurality of compounds of formula (I)” means more than one compound of formula (I), wherein each of the compounds of formula (I) has the same structure.

In one embodiment the invention provides a composition comprising a plurality of compounds of formula (I) and one or more lipids.

As used herein, the term “encapsulate” means an aggregate, having a molecule (e.g., a therapeutic agent) surrounded or partially surrounded by a plurality of compounds of formula (I). In certain embodiments, the term “encapsulate” means an aggregate, having a molecule (e.g., a therapeutic agent) surrounded or partially surrounded by a plurality of compounds of formula (I) and one or more lipids.

As used herein, the term “stabilized encapsulate” means an aggregate, having a molecule (e.g., a therapeutic agent) surrounded or partially surrounded by a plurality of compounds of formula (I). In certain embodiments, the term “stabilized encapsulate” means an aggregate, having a molecule (e.g., a therapeutic agent) surrounded or partially surrounded by a plurality of compounds of formula (I) and one or more lipids.

As used herein, the phrase “low-density lipoprotein (LDL)” includes “unoxidized LDL,” “weakly oxidized LDL” and “oxidized LDL.” LDLs bind to proteoglycans (PGs), the major low density lipoprotein (LDL)-retentive matrix molecules within the vascular intima are proteoglycans. LDL binding to PGs modifies the LDL surface, rendering the LDL susceptible to oxidation induced by Cu²⁺ and macrophages. The oxidative modification of LDL lowers its localized positive charge relative to native LDL, thus reducing the affinity of LDL for anionically charged PGs. The increase in the net negative charge on oxidized LDL also leads to the reduced recognition of oxidized LDL by the classical LDL receptor, and increased recognition by the scavenger receptors on macrophages in the intima. Thus, “unoxidized low-density lipoprotein” refers to a native LDL, e.g., an LDL that has the characteristics of an LDL that is recognized by a native LDL receptor. In contrast, an “oxidized LDL (ox-LDL)” is a modified LDL recognized by scavenger receptors. By the phrase “weakly oxidized low-density lipoprotein (LDL)” is meant a mildly or partially oxidized LDL. Both unoxidized and weakly oxidized LDL have relatively high localized positive charges, e.g., due to unmodified Lys and Arg residues on apolipoprotein B-100 (ApoB-100) (LDL have a single Apo B-100 molecule on their surface) as compared to oxidized LDL. See, for example, Chnari et al., Biomaterials, 26: 3749-3758 (2005) and Chnari et al., Biomacromolecules. 2006 Feb;7(2):597-603.

By “reduction” is meant the separation or removal (e.g., lowered concentration of a substance, such as LDL) from a physiological sample or the blood stream of a subject. For example, in one embodiment of the invention, a compound of formula I is administered to a patient and becomes associated with LDL in a manner that will provide a beneficial physiological effect. For example, it is possible that the compound of formula I may cause certain forms of the LDL to be eliminated from a subject, or prevent other forms of LDL from having physiological and/or pathological activity. In certain embodiments, the compound of formula (I) may attach itself to LDL and cause the LDL to be eliminated from a subject, or prevent other forms of LDL from having physiological and/or pathological activity.

For example, it is also possible that the compound of formula I can inhibit the uptake of modified forms of LDL mediated by scavenger receptors (e.g., scavenger receptor A (SR-A) or CD36) and counteract cholesterol accumulation and foam cell formation, characteristics of the onset of atherogenesis. In certain embodiments, a compound of formula I competitively inhibits scavenger receptor-mediated LDL uptake. In certain embodiments, a compound of formula I competitively inhibits scavenger receptor-mediated LDL uptake in macrophages. In certain embodiments the scavenger receptor is SR-A. In certain embodiments the LDL is oxLDL.

Certain embodiments of the invention provide a method for preventing the uptake of LDL by a cell (e.g., a macrophages or smooth muscle cells), comprising contacting the cell with a compound of formula I as described herein.

In certain embodiments, the cell expresses a scavenger receptor (e.g., SR-A or CD36).

In certain embodiments, a compound of formula (I) interacts with the scavenger receptor. In certain embodiments, a compound of formula (I) binds to the scavenger receptor.

By “inhibition of atherosclerotic development” is meant the suppression of the development, progression and/or severity of atherosclerosis, a slowly progressive disease characterized by the accumulation of cholesterol within the arterial wall, e.g. by inhibiting, preventing or causing the regression of an atherosclerotic plaque.

When a plurality of compounds of formula (I) are placed in a hydrophilic solvent (e.g., an aqueous solution comprising water or wherein the solvent is water), Applicant has discovered that the compounds of formula (I) will aggregate, with the polyether portion of the compounds extending into the hydrophilic solvent, and the hydrophobic chain portions of the compounds forming a hydrophobic core. Such aggregates can solubilize a hydrophobic molecule (e.g., a hydrophobic therapeutic agent) in the aqueous solvent, by encapsulating the hydrophobic molecule in the hydrophobic core of the aggregates. The hydrophobic molecule can typically be added to the solution of the compounds of formula (I) subsequent to aggregation, or the hydrophobic molecule can be added to the solution of the compounds of formula (I) prior to aggregation, allowing the aggregates to form around the molecule. Thus, the aggregates formed from the compounds of formula (I) can function similar to traditional micelles.

Typically, the aggregates of the invention have a diameter of from about 10 nm to about 1000 nm. The diameters can be measured using any suitable analytical technique, such as, for example, dynamic light scattering.

Compounds of formula (I) can be used to form aggregates that function similar to conventional “micelles”. These aggregates can be used for essentially any application in which conventional micelles are employed. Examples include drug solubilization, fragrance encapsulation, passive targeting for drug delivery, waste water treatment, enhanced capillary electrophoresis activation, and induction of protein crystallization.

Accordingly, as used herein, the term “molecule” includes any compound that can be incorporated into an aggregate as described herein. Typically, “molecules” have solubility properties that are undesirable and that can be modified by incorporation into an aggregate of the invention. For example, the term “molecule” includes therapeutic agents, insecticides, pesticides, herbicides, antiseptics, food additives, fragrances, dyes, diagnostic aids, and the like. Other specific examples of molecules include, but are not limited to:

abietic acid, aceglatone, acenaphthene, acenocoumarol, acetohexamide, acetomeroctol, acetoxolone, acetyldigitoxins, acetylene dibromide, acetylene dichloride, acetylsalicylic acid, alantolactone, aldrin, alexitol sodium, allethrin, allylestrenol, allyl sulfide, alprazolam, aluminum bis(acetylsalicylate), ambucetamide, aminochlothenoxazin, aminoglutethimide, amyl chloride, androstenediol, anethole trithone, anilazine, anthralin, Antimycin A, aplasmomycin, arsenoacetic acid, asiaticoside, astemizole, aurodox, aurothioglycanide, 8-azaguanine, azobenzene;

baicalein, Balsam Peru, Balsam Tolu, barban, baxtrobin, bendazac, bendazol, bendroflumethiazide, benomyl, benzathine, benzestrol, benzodepa, benzoxiquinone, benzphetamine, benzthiazide, benzyl benzoate, benzyl cinnamate, bibrocathol, bifenox, binapacryl, bioresmethrin, bisabolol, bisacodyl, bis(chlorophenoxy)methane, bismuth iodosubgallate, bismuth subgallate, bismuth tannate, Bisphenol A, bithionol, bomyl, bromoisovalerate, bomyl chloride, bomyl isovalerate, bornyl salicylate, brodifacoum, bromethalin, broxyquinoline, bufexamac, butamirate, butethal, buthiobate, butlated hydroxyanisole, butylated hydroxytoluene;

calcium iodostearate, calcium saccharate, calcium stearate, capobenic acid, captan, carbamazepine, carbocloral, carbophenothin, carboquone, carotene, carvacrol, cephaeline, cephalin, chaulmoogfic acid, chenodiol, chitin, chlordane, chlorfenac, chlorfenethol, chlorothalonil, chlorotrianisene, chlorprothixene, chlorquinaldol, chromonar, cilostazol, cinchonidine, citral, clinofibrate, clofazimine, clofibrate, cloflucarban, cionitrate, clopidol, clorindione, cloxazolam, coroxon, corticosterone, coumachlor, coumaphos, coumithoate cresyl acetate, crimidine, crifomate, cuprobam, cyamemazine, cyclandelate, cyclarbamate cymarin, cypennethril;

dapsone, defosfamide, deltamethrin, deoxycorticocosterone acetate, desoximetasone, 10 dextromoramide, diacetazoto, dialifor, diathymosulfone, decapthon, dichlofluani, dichlorophen, dichlorphenamide, dicofol, dicryl, dicmarol, dienestrol, diethylstilbestrol, difenamizole, dihydrocodeinone enol acetate, dihydroergotamine, dihydromorphine, dihydrotachysterol, dimestrol, dimethisterone, dioxathion, diphenane, N-(1,2-diphenylethyDnicofinamide, dipyrocetyl, disulfamide, dithianone, doxenitoin, drazoxolon, durapatite, edifenphos, emodin, enfenamic acid, erbon, ergocorninine, erythrityl tetranitrate, erythromycin stearate, estriol, ethaverine, ethisterone, ethyl biscomacetate, ethyihydrocupreine, ethyl menthane carboxamide, eugenol, euprocin, exalamide;

febarbamate, fenalamide, fenbendazole, fenipentol, fenitrothion, fenofibrate, fenquizone, fenthion, feprazone, flilpin, filixic acid, floctafenine, fluanisone, flumequine, fluocortin butyl, fluoxymesterone, flurothyl, flutazolam, fumagillin, 5-furfuryl-5-isopropylbarbitufic acid, fusafungine, glafenine, glucagon, glutethimide, glybuthiazole, griseofulvin, guaiacol carbonate, guaiacol phosphate, halcinonide, hematoprphyrin, hexachlorophene, hexestrol, hexetidine, hexobarbital, hydrochlorothiazide, hydrocodone, ibuproxam, idebenone, indomethacin, inositol niacinate, iobenzamic acid, iocetamic acid, iodipamide, iomeglamic acid, ipodate, isometheptene, isonoxin, 2-isovalerylindane- 1,3-dione;

josamycin, 11-ketoprogesterone, laurocapram, 3-0-lauroylpyridoxol diacetate, lidocaine, lindane, linolenic acid, liothyronine, lucensomycin, mancozeb, mandelic acid, isoamyl ester, mazindol, mebendazole, mebhydroline, mebiquine, melarsoprol, melphalan, menadione, menthyl valerate, mephenoxalone, mephentermine, mephenytoin, meprylcaine, mestanolone, mestranol, mesulfen, metergoline, methallatal, methandriol, methaqualone, 3-methylcholanthrene, methylphenidate, 17-methyltestosterone, metipranolol, minaprine, myoral, nafialofos, nafiopidil, naphthalene, 2-naphthyl lactate, 2-(2-naphthyloxy)ethan01, naphthyl salicylate, naproxen, nealbarbital, nemadectin, niclosamide, nicoclonate, nicomorphine, nifuroquine, nifuroxazide, nitracrine, nitromersol, nogalamycin, nordazepam, norethandrolone, norgestrienone;

octavefine, oleandrin, oleic acid, oxazepam, oxazolam, oxeladin, oxwthazaine, oxycodone, oxymesterone, oxyphenistan acetate, paraherquamide, parathion, pemoline, pentaerythritol tetranitrate, pentylphenol, perphenazine, phencarbamide, pheniramine, 2-phenyl-6-chlorophenol, phentlmethylbarbituric acid, phenytoin, phosalone, phthalylsulfathiazole, phylloquinone, picadex, pifarnine, piketopfen, piprozolin, pirozadil, plafibride, plaunotol, polaprezinc, polythiazide, probenecid, progesterone, promegestone, propanidid, propargite, propham, proquazone, protionamide, pyrimethamine, pyrimithate, pyrvinium pamoate; quercetin, quinbolone, quizalofo-ethyl, rafoxanide, rescinnamine, rociverine, ronnel salen, scarlet red, siccmn, simazine, simetfide, sobuzoxane, solan, spironolactone, squalene, stanolone, sucralfate, sulfabenz, sulfaguanole, sulfasalazine, sulfoxide, sulpiride, suxibuzone, talbutal, terguide, testosterone, tetrabromocresol, tetrandrine, thiacetazone, thiocolchicine, thiocftc acid, thioquinox, thioridazine, thiram, thymyl N-isoamylcarbamate, tioxidazole, tioxolone, tocopherol, tolciclate, tolnafiate, triclosan, triflusal, triparanol;

ursolic acid, valinomycin, verapamil, vinblastine, vitamin A, vitamin D, vitamin E, xenbucin, xylazine, zaltoprofen, and zearalenone.

The aggregates of the invention are particularly useful for solubilizing hydrophobic molecules, particularly therapeutic agents that are hydrophobic in nature. Thus, according to one embodiment of the present invention, a therapeutic agent is encapsulated by combining the agent and a plurality of compounds of formula (I) in a solvent, such as water. The present invention contemplates the use of encapsulated hydrophobic molecules at concentrations ranging from 10⁻³ to 10⁻⁶ M. At the same time, another advantage of the present invention is the thermodynamic stability of the polymers, which permit the formation of low concentration stable aqueous solutions of the polymer encapsulates, far below the CMC's of conventional surfactants. CMC values range from 10⁻⁴to 10⁻⁷ M but may be as low as 10⁻¹⁰ which is below the limits of detection. CMC is the critical micellar concentration, the concentration at which a majority of the polymers are comprised within micellar aggregates vs. individual polymer chains.

The encapsulates of the invention that comprise a therapeutic agent can be formulated as pharmaceutical compositions and administered to a mammalian host, such as a human patient in a variety of forms adapted to the chosen route of administration, i.e. parenterally, by intravenous, intramuscular, topical or subcutaneous routes. Thus, the encapsulates of the invention may be systemically administered, in combination with a pharmaceutically acceptable vehicle such as an inert diluent.

The encapsulates of the invention may be administered intravenously or intraperitoneally by infusion or injection. Solutions of the encapsulates can be prepared, for example, in water. Under ordinary conditions of storage and use, these preparations may contain a preservative to prevent the growth of microorganisms.

The pharmaceutical dosage forms suitable for injection or infusion should be sterile, fluid and stable under the conditions of manufacture and storage. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride.

Sterile injectable solutions are prepared by incorporating the encapsulates of the invention in the required amount in the appropriate solvent with various of the other ingredients enumerated above, as required, followed by sterilization.

The dose and method of administration will vary from animal to animal and be dependent upon such factors as the type of animal being treated, its sex, weight, diet, concurrent medication, overall clinical condition, the particular therapeutic agent employed, the specific use for which the agent is employed, and other factors which those skilled in the relevant field will recognize.

Therapeutically effective dosages may be determined by either in vitro or in vivo methods. For each particular dosage form of the present invention, individual determinations may be made to determine the optimal dosage required. The range of therapeutically effective dosages will naturally be influenced by the route of administration, the therapeutic objectives, and the condition of the patient. The determination of effective dosage levels, that is, the dosage levels necessary to achieve the desired result, will be within the ambit of one skilled in the art. Typically, applications of agent are commenced at lower dosage levels, with dosage levels being increased until the desired effect is achieved.

A typical dosage might range from about 0.001 mg to about 1,000 mg of therapeutic agent, per kg of animal weight. Preferred dosages range from about 0.01 mg/kg to about 100 mg/kg, and more preferably from about 0.10 mg/kg to about 20 mg/kg. Advantageously, the dosage forms of this invention may administered several times daily, and other dosage regimens may also be useful.

According to the invention, aggregate degradation is not a prerequisite for release of the molecule (e.g. the therapeutic agent).

The compounds of formula (I), aggregates and encapsulates of the invention may also be used as thickening agents, lubricants, detergents surfactants, plasticizers and anti-fouling agents. The compounds of formula (I), aggregates and encapsulates of the invention may be used as an emulsifying, dispersing or stabilizing agent for dyes, cosmetics, pigment and pharmaceutical products. The compounds of formula (I), aggregates and encapsulates of the invention are particularly useful as an, emulsifying, dispersing or stabilizing agent in the dyeing of textiles and for encapsulating dyes, fragrances, or both for cosmetics. The compounds of formula (I), aggregates and encapsulates of the invention are useful as lubricants and as a thickening agents for paints. The compounds of formula (I), aggregates and encapsulates of the invention may also be employed as an emulsifying, dispersing or stabilizing agent for components of photographic compositions and developers.

For therapeutic applications, the preferred aggregates of the invention hydrolyze into components known to be biocompatible, i.e., sugars, fatty acids, amino acids and poly(ethylene glycol). This also results in low cytotoxicity of the polymer and its hydrolysis products. The poly(alkylene oxide) units enhance the immunogenicity of the encapsulate, enabling the hydrophobic molecules to evade the body's immune system, thereby increasing the circulation time of the hydrophobic molecule. This allows for effective treatment with reduced quantities of the hydrophobic molecule, which, together with the enhanced immunogenicity, prevents or reduces the severity of incidents of toxic side effects.

The following non-limiting examples set forth herein below illustrate certain aspects of the invention.

### EXAMPLES

Amphiphilic macromolecules (AMs) based on carbohydrate domains functionalized with poly(ethylene glycol) can inhibit the uptake of oxidized low density lipoprotein (oxLDL) mediated by scavenger receptor A (SR-A) and counteract foam cell formation, the characteristic “atherosclerotic” phenotype. AMs generated from compound 2, discussed below, were found to inhibit oxLDL uptake in cell lines expressing SR-A.

Atherosclerosis, the occlusive artery disease, is triggered by the build-up of oxidized low density lipoprotein (oxLDL) in blood vessel walls. The oxLDL accumulation generates an inflammatory response which results in the recruitment of circulating monocytes followed by their differentiation to macrophages and upregulation of macrophage membrane-based scavenger receptors (Yoshimoto et al., Advances in experimental medicine and biology, 2002; 507:403-7). The major scavenger receptors, namely scavenger receptor A (SR-A) and CD36, mediate the uptake of oxLDL, (Goldstein et al., Proc Natl Acad Sci USA 1979; 76:333-7; Podrez et al., J Clin Invest 2000; 105:1095-108; de Winther et al., Arteriosclerosis, thrombosis, and vascular biology 2000; 20:290-7) leading to unregulated cholesterol accumulation and foam cell formation, a key characteristic of the onset of atherogenesis (Brown et al., Annual review of biochemistry 1983; 52:223-61; Steinberg, J Biol Chem 1997; 272:20963-6).

Aside from lifestyle changes, cholesterol lowering therapies (i.e. statins) are the most common methods for atherosclerosis treatment. Such drugs, however, indirectly mediate atherosclerosis by decreasing cholesterol synthesis which ultimately results in decreased oxLDL in the blood vessel walls. A more direct and promising approach in the treatment and prevention of atherosclerosis involves designing functional inhibitors against scavenger receptors to abrogate uncontrolled oxLDL uptake. Carbohydrate-based, nanoscale amphiphilic macromolecules (AMs) that competitively inhibit scavenger receptor-mediated oxLDL uptake have previously been described (Chnari et al., Biomacromolecules 2006; 7:597-603; Chnari et al., Biomaterials 2005; 26:3749-58). Comprised of a hydrophobic domain based on the sugar, mucic acid, acylated with four aliphatic chains, and a hydrophilic poly(ethylene glycol) (PEG) tail, the AMs exhibit high biocompatibility and stability (Tian et al., Macromolecules 2004; 37:538-43). Further, the AMs self-assemble into micelles in aqueous media at relatively low (10⁻⁴ M) concentrations, enabling drug encapsulation within the micellar core. To understand the key structural features relevant to oxLDL inhibition, systematic variations to the AM structure were performed, including the PEG chain length, PEG architecture and aliphatic chain length as well as type, charge, number, and rotational motion of anionic charges (Plourde et al., Biomacromolecules 2009; 10:1381-91; Iverson et al., Acta Biomater 2010; 6:3081-91).

The AM hydrophobic domain compositions and backbone carbohydrate stereochemistry and conformation can also regulate the activity of the AMs. To this end, several AMs were synthesized with incremental backbone modifications to investigate the influence of carbohydrate stereochemistry, aromatic groups and cyclic carbohydrates that are incorporated within the polymer backbone on biological activity, specifically, oxLDL inhibition. To confirm the extent of backbone influence on scavenger receptor binding, a functional assay was developed. The AMs were evaluated for their ability to inhibit SR-A mediated oxLDL uptake in human embryonic kidney cells expressing the SR-A scavenger receptor (HEK-SRA). These studies demonstrated that minor changes in the AMs architecture can significantly affect the physicochemical properties and inhibition of oxLDL uptake. One of the developed AMs, generated from compound 2, is described below in the following Examples.

All parts and percentages are by weight unless otherwise noted and all temperatures are in degrees Celsius.

All PEG's were obtained from Shearwater Polymers (Birmingham, Ala.) and used without further purification. All other chemicals were obtained from Aldrich (Milwaukee, Wis.), and used without further purification. Analytical grade solvents were used for all the reactions. Methylene chloride, tetrahydrofuran (THF), triethylamine (TEA) and dimethylsulfoxide (DMSO) were distilled.

¹H-NMR spectra were obtained using a Varian 400 MHz or 500 MHz spectrophotometer with TMS as internal reference. Samples were dissolved in CDCl₃, or CDCl₃ with a few drops of DMSO-d₆ if necessary. Molecular weights (MW) were determined using gel permeation chromatography (GPC) with respect to PEG standards (Sigma-Aldrich) on a Waters Stryagel® HR 3 THF column (7.8×300 mm). The Waters LC system (Milford, Mass.) was equipped with a 2414 refractive index detector, a 1515 isocratic HPLC pump, and 717plus autosampler. Samples (10 mg/mL) were dissolved in THF and filtered using 0.45 gm pore size nylon or PTFE syringe filters (Fisher Scientific). Dynamic light scattering (DLS) analysis was carried out on a Zetasizer nanoseries ZS90 (Malvern instruments). CMC studies were carried out on a Spex fluoromax-3 spectrofluorometer (Jobin Yvon Horiba) at 25° C. Melting points were determined by DSC on a TA DSC Q200. TA universal analysis 2000 software was used for data collection on a Dell dimension 3000 computer. Samples (4-8 mg) were heated under dry nitrogen gas. Data were collected at heating and cooling rates of 10° C. min⁻¹with a two-cycle minimum.

Compounds of formula 1 can be prepared as illustrated in Scheme 1. Compounds of formula 1 with different hydrophobic chains or different fatty acid residues, polyether moieties, number of hydroxy groups on the aryl or heteroaryl and/or different Y—Z values can also be prepared by this same general procedure. Compounds of formula 1 wherein the aryl or heteroaryl are substituted with amine groups, or a mixture of amine and hydroxyl groups, which hydroxy and amine groups may be subsequently acylated can also prepared by this route.

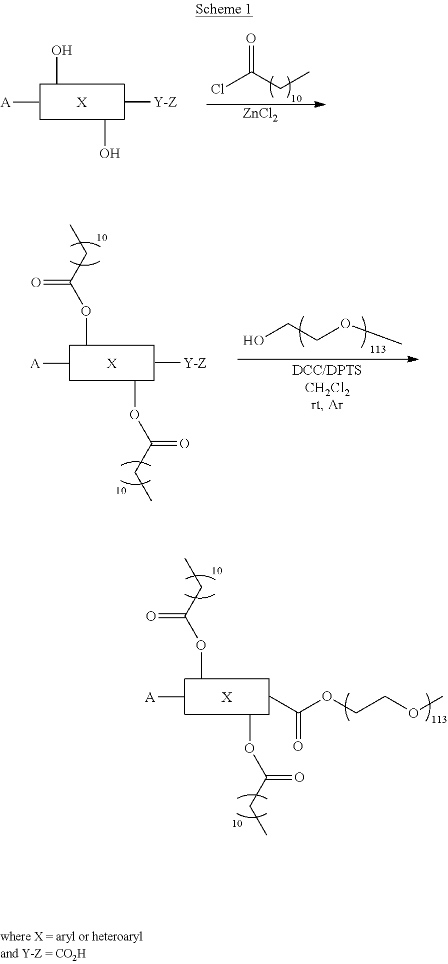

### Example 1

### Preparation of Compound 2

Preparation of Compound 1:

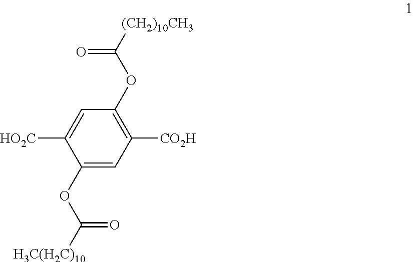

2,5-Dihydroxyterephthalic acid was chosen as the building block as it possesses both two carboxylic acids and two hydroxyl groups for functionalization.

2,5-Dihydroxyteraphthalic acid (5.0 g, 25 mmol) and zinc chloride (0.81 g, 5.9 mmol) were stirred with lauroyl chloride (87 ml, 378 mmol). The solution was heated to 95° C. in a temperature-controlled oil bath and stirred under argon overnight. Diethylether (100 ml) and water (30 ml) were added and the solution was allowed to stir for 45 minutes. The organic phase was washed with water (5×100 ml) and then concentrated via rotary evaporation. The concentrated solution was precipitated from hexanes (2.5 L) yielding 1. (10.1 g, 68%). ¹H NMR (CDCl₃): 0.80-0.93 (t, 6H, CH₃), 1.20-1.45 (m, 32H, CH₂), 1.70-1.81 (m, 4H, CH₂), 2.51-2.65 (m, 4H, CH₂), 7.70 (s, 2H, Ar—CH); ¹³C NMR (CDC₁₃): 14.32, 22.87, 29.34, 29.53, 29.67, 29.80, 29.82, 32.10, 34.37, 127.37, 128.74, 147.84, 165.27, 172.42; IR (NaCl cm⁻¹): 2847, 1768, 1690 (C═O), 1268, 1177, 935, 897; Tm=71° C.

Preparation of Compound 2:

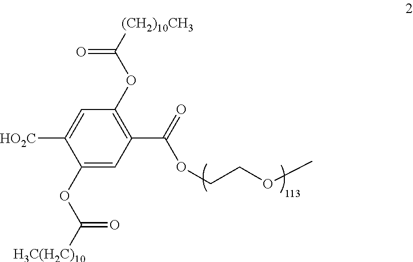

PEG (1.0 g, 0.2 mmol) was azeotropically distilled with toluene. Compound 1 (0.4 g, 0.7 mmol) and DPTS (0.1 g, 0.3 mmol) were dissolved in anhydrous CH₂Cl₂ (15 ml). PEG was cooled to room temperature under argon and the solution of 1 and DPTS was added. Once the PEG had dissolved, dicyclohexylcarbodiimide (DCC) (1 ml, 1.0 mmol) was added dropwise. The reaction mixture was stirred under argon for 48 hours, cooled and the resulting white solid precipitate (dicyclohexylurea) was removed by vacuum filtration. The filtrate was washed with 0.1N HCl (20 ml), followed by 50:50 brine:water (2×20 ml), dried over MgSO₄ and concentrated via rotary evaporation. The product was precipitated from CH₂Cl₂ and diethyl ether yielding 2 as a white solid. (1.0 g 91%) ¹H NMR (CDCl₃): δ 0.85 (t, 6H, CH₃), 1.30 (m, 32H, CH₂), 1.71 (m, 4H, CH₂), 2.21 (m, 4H, CH₂), 2.36 (m, 4H, CH₂), 3.63 (m,˜0.4-kH, CH₂), 7.65 (d, 1H, Ar—H), 8.10 (s, 1H, Ar-H); IR (NaCl cm⁻¹): 3426, 2879, 1650 (C═O), 1108, 949, 842; Tm=56° C.; GPC: M_{w}=6.3 kDa; PDI=1.0.

### Example 2

### CMC Measurement

A solution of pyrene, the fluorescence probe molecule, was made up to a concentration of 5×10⁻⁶M in acetone. Samples were prepared by adding 1 mL of pyrene solution to a series of vials and allowing the acetone to evaporate so that the final concentration of pyrene in all of the samples was 5×10⁻⁷ M. AMs were dissolved in HPLC grade water and diluted to a series of concentrations from 1×10⁻³ M to 1×10⁻¹⁰ M. AM-pyrene solutions (10 mL) were shaken overnight at 37° C. to allow partition of the pyrene into the micelles. Excitation was performed from 300 to 360 nm, with 390 nm as the emission wavelength. The maximum absorption of pyrene shifted from 332 to 334.5 nm on micelle formation (Astafieva et al., Macromolecules 1993; 26:7339-52; Meng et al., J Appl Polym Sci 2009; 114:2195-203; Kalyanasundaram et al., Am Chem Soc 1977; 99:2039-44). The ratio of absorption of encapsulated pyrene (334.5 nm) to pyrene in water (332 nm) was plotted as the logarithm of polymer concentrations. The inflection point of the curve was taken as the CMC.

The physicochemical properties (hydrodynamic diameter, melting temperature (Tₘ) and critical micelle concentration (CMC)) were evaluated. The Tₘ value of compound 2 was 56° C. and the other synthesized polymers also had Tₘ values of ˜60° C. This demonstrated that the PEG domain dominates the polymer's thermal behavior. Previous work indicated that the AM biological activity was dependent on their ability to form stable micelles (Plourde et al., Biomacromolecules 2009; 10:1381-91; Iverson et al., Acta Biomater 2010; 6:3081-91). The AMs with the lowest CMC values are extremely efficacious and the change in CMC may have a biological impact for the newly synthesized AMs. CMC values were measured using a previously reported fluorimetry technique using pyrene as the fluorescence probe (Astafieva et al., Macromolecules 1993; 26:7339-52; Meng et al., J Appl Polym Sci 2009; 114:2195-203; Kalyanasundaram et al., J Am Chem Soc 1977; 99:2039-44). Incorporation of the rigid, aromatic 2,5-dihydroxyterephthalic acid backbone of compound 2 yielded micelles almost twice the size (˜35 nm) of the linear analogs, but with a comparable CMC value (10⁻⁷M) (see Table 1 below). The increase in size was anticipated as terephthalic acid is more sterically bulky than the linear carbohydrates. The CMC, size and Tm of compound 2 is shown in Table 1.

TABLE 1
Size, CMC and Tm

| Test | Size | CMC | Tm |
|---|---|---|---|
| Compound | Z-Ave (nm) | M | ° C. |
| 2 | 35 | 1.6 × 10⁻⁷ | 56 |

### Example 3

### Cell Culture

### Methods

### Cell Culture

Studies of polymer interactions were conducted using a tet-inducible cell line with controlled expression of scavenger receptor A (SR-A), human embryonic kidney (HEK) cells stably transfected with human SR-A (gift from Dr. Steven R. Post), which are referred to as HEK-SRA. Cells were propagated in high glucose Dulbecco's Modified Eagle Medium (DMEM) (Invitrogen) supplemented with 10% fetal bovine serum (FBS), 1% penicillin/streptomycin, 15 μg/mL Blasticidin and 100 μg/mL HygromycinB at 37° C. in 5% CO₂. SR-A expression was induced with addition of 0.5 μg/ml tetracycline overnight and throughout the experiment.

### LDL Oxidation

Oxidized low density lipoprotein (oxLDL) was generated by incubating 50 μg/ml LDL purified from human plasma (Molecular Probes Eugene, OR) with 10 μM CuSO₄ at 37° C. for 18 hr exposed to air (Chang et al., J Lipid Res 2001; 42:824-33; Oorni et al., J Biol Chem 1997; 272:21303-11). Oxidation was terminated with 0.01% w/v EDTA (Sigma, St. Louis, Mo.).

### OxLDL Accumulation in HEK-SRA Cells

The internalization of oxLDL by HEK-SRA cells was assayed by incubating borondipyrromethene (BODIPY)-labeled oxLDL (10 μg/ml) with cells for 24 hr at 37° C. and 5% CO₂ in serum containing DMEM. Conditions included a control of medium alone without polymer intervention, and non-induced cells. Cells were washed with lx PBS, fixed with 4% formaldehyde and imaged on a Nikon Eclipse TE2000-S fluorescent microscope to determine fluorescently tagged oxLDL accumulation. The images were analyzed with ImageJ 1.42q (NIH) and fluorescence data was normalized to cell count. The levels of oxLDL uptake were normalized to those obtained in the absence of polymers.

### Statistical Analysis

Each in vitro experiment was performed at least twice and three replicate samples were investigated in each experiment. Five images per well were captured and analyzed. The results were then evaluated using analysis of variance (ANOVA). Significance criteria assumed a 95% confidence level (P<0.05). Standard error of the mean is reported in the form of error bars on the graphs of the final data.

### Results

The AMs were evaluated for their ability to inhibit oxLDL internalization through in vitro structure-activity relationship studies with HEK-SRA cells. In vitro experiments were carried out via incubation of the cells with 10⁻⁶ M polymers and fluorescently labeled oxLDL for 24 hr at 37° C. As controls, the basal uptake of oxLDL when SRA-expression was not induced and the basal uptake of oxLDL when no polymer was present were both evaluated.

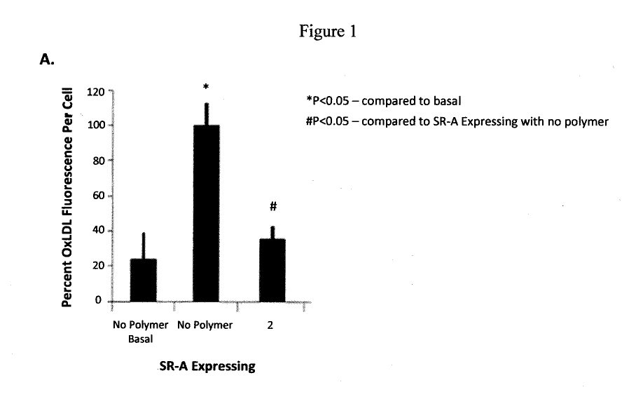

The AMs generated from compound 2 inhibited ˜60% oxLDL uptake in HEK-SRA cells (FIG. 1A). Evaluation of the modified polymers indicated that the ability to bind to HEK-SRA cells does not correlate to the solution stability of the AMs. The data suggested that the polymer behavior may be largely dominated by the unimer, such that the individual polymer chains govern binding to scavenger receptors.

As indicated above, a series of AMs were designed to investigate the influence of hydrophobic domain stereochemistry and composition on aggregation and biological properties. While seemingly small with respect to the overall polymer compositions, minor alterations in backbone architecture resulted in differences in the physicochemical properties of the AMs, particularly in micelle size and solution stability. In vitro experiments were implemented to examine AM binding to SR-A. These studies also demonstrated that minute changes in the polymer structure affect SR-A binding affinities and consequently modulate the competitive inhibition of oxLDL uptake. These findings establish that the composition of the hydrophobic domain is an important design factor for these polymers as candidates for cardiovascular therapeutics.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula (I):
A-X—Y—Z—R₁ (I)
wherein;
A is an anionic group or is absent;
X is an aryl or heteroaryl, wherein any aryl or hetereoaryl of X is substituted with one or more groups selected from hydroxy and amine, wherein one or more of the hydroxy or amine groups is acylated with a hydrophobic chain, and wherein any aryl or hetereoaryl of X is optionally substituted with one or more groups selected from halo, carboxy, CN, NO₂, N((C₁-C₆)alkyl)₂, (C₁-C₆)alkyl, (C₁-C₆)alkoxy, CF₃ and OCF₃;
Y is —C(═O)—, —C(═S)—, or is absent;
Z is O, S or NH; and
R₁ is a polyether,
or a salt thereof.

2. The compound of claim 1 wherein X is phenyl substituted with two or more hydroxy groups, wherein two or more of the hydroxy groups are acylated with a hydrophobic chain.

3. The compound of claim 1 wherein the A is selected from carboxy, -SO₃H and —PO₃H.

4. The compound of claim 1 wherein the Y is —C(═O)—.

5. The compound of claim 1 wherein the Z is O.

6. The compound of claim 1 wherein the Y—Z is —C(═O)NH— or —C(═O)O—.

7. The compound of claim 1 wherein the polyether is a poly(alkylene oxide) having between about 2 and about 150 repeating units.

8. The compound of claim 1 wherein the polyether has the following structure:
R₅—(R₆—O—)ₐ—R₆—
wherein R₅ is a 1 to 20 carbon straight-chain or branched alkyl group, —OH, —OR₇, —NH₂, —NHR₇, —NHR₇R₈, —CO₂H, —SO₃H (sulfo), —CH₂—OH, —CH₂—OR₇, —CH₂—O—CH₂—R₇, —CH₂—NH₂, —CH₂—NHR₇, —CH₂—NR₇R₈, —CH₂CO₂H, —CH₂SO₃H, or —O—C(═O)—CH₂—CH₂—C(═O)—O—;
R₆ is a 1 to 10 carbon straight-chain or branched divalent alkylene group;
each R₇ and R₈ is independently a 1 to 6 carbon straight-chain or branched alkylene group; and
a is an integer from 2 to 150, inclusive.

9. The compound of claim 1 wherein the hydrophobic chain comprises (C₁-C₂₄)alkyl, (C₂-C₂₄)alkene or (C₂-C₂₄)alkyne.

10. The compound of claim 1 wherein the hydrophobic chain is a fatty acid, wherein the fatty acid is caprylic, capric, lauric, myristic, myristoleic, palmitic, palmitoleic, stearic, oleic, linoleic, arachidic, behenic, or erucic acid, or a mixture thereof

11. The compound of claim 1, wherein the compound is:

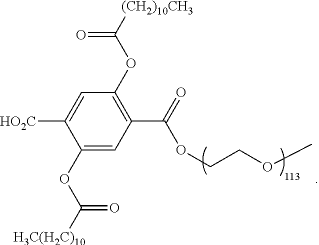

12. A composition comprising a plurality of compounds of formula (I) as described in claim 1 in a solvent.

13. An aggregate structure formed by combining a plurality of compounds of formula (I), as described in claim 1, in a solvent; and allowing the compounds to form the aggregate.

14. A method for preparing an aggregate structure of compounds of formula (I), as described in claim 1, comprising combining a plurality of compounds of formula (I) in a solvent; and allowing them to form the aggregate structure.

15. An encapsulate comprising a molecule surrounded or partially surrounded by a plurality of compounds of formula (I), as described in claim 1.

16. A method for preparing an encapsulate as described in claim 15, comprising combining a plurality of compounds of formula (I), as described in claim 1, and a molecule in a solvent; and allowing the compounds of formula (I) to aggregate around the molecule, to provide the encapsulate.

17. A composition comprising a solvent, and an aggregate of a plurality of compounds of formula (I) as described in claim 1 surrounding a molecule.

18. A pharmaceutical composition comprising an encapsulate as described in claim 15, or a pharmaceutically acceptable salt thereof, and a pharmaceutically acceptable carrier.

19. A method for preventing the uptake of LDL by a cell comprising contacting the cell with a compound of formula I as described in claim 1.

20. A method for inhibiting atherosclerosis or atherosclerotic development in a mammal, comprising administering an anti-atherosclerosis or anti-atherosclerotic development amount of a compound of formula I as described in claim 1, or a pharmaceutically acceptable salt thereof, to the mammal.
